(19) United States
(12) Patent Application Publication
(10) Pub. No.: US 2016/0166856 A1
(43) Pub. Date: Jun. 16, 2016

(54) PREDICTIVE COLLISION AVOIDANCE FOR RADIOTHERAPY

(71) Applicant: THE UAB RESEARCH FOUNDATION, Birmingham, AL (US)

(72) Inventors: RICHARD POPPLE, HOMEWOOD, AL (US); REX CARDAN, HOOVER, AL (US)

(21) Appl. No.: 14/908,783

(22) PCT Filed: Jul. 31, 2014
(86) PCT No.: PCT/US14/49078

§ 371 Date: Jan. 29, 2016

Related U.S. Application Data
(60) Provisional application No. 61/860,561, filed on Jul. 31, 2013.

(51) Int. Cl. A61N 5/10

(52) U.S. Cl. CPC A61N 5/1048; A61N 5/1081

(57) ABSTRACT

Disclosed are various embodiments for predicting and avoiding collisions during radiotherapy. A depth map produced by at least one three-dimensional camera is obtained by a computing device. The computing device identifies a plurality of objects in the depth map, wherein the plurality of objects comprise a radiation therapy machine and a patient. The computing device generates a corresponding three-dimensional model for each one of the plurality of objects. The computing device then determines whether the corresponding three-dimensional model for each one of the plurality objects overlaps with another corresponding three-dimensional model for another one of the plurality objects.

### CROSSREFERENCE TO RELATED APPLICATIONS

This application claims priority to U.S. Provisional Application 61/860,561, filed on Jul. 31, 2013, which is incorporated by reference herein in its entirety.

### BACKGROUND

Moving radiation therapy equipment can collide with objects in the treatment room, including patients, during radiation treatment therapy procedures. This can result in costly damage to radiation therapy machines as well as injury or death to patients.

### SUMMARY

Disclosed are various embodiments for a non-transitory computer-readable medium embodying a program executable in at least one computing device, comprising: code that obtains a depth map produced by a three-dimensional camera; code that identifies a plurality of objects in the depth map, wherein the plurality of objects comprise a radiation therapy machine and a patient; code that generates a corresponding three-dimensional model for each one of the plurality of objects; and code that determines whether the corresponding three-dimensional model for each one of the plurality objects overlaps with another corresponding three-dimensional model for another one of the plurality objects. In some embodiments, the program further comprises: code that obtains an updated depth map produced by the at least one three-dimensional camera; code that identifies the plurality of objects in the updated depth map; and code that determines, based at least in part on the updated depth map, whether the corresponding three-dimensional model for each one of the plurality of objects overlaps with another corresponding three-dimensional model for another one of the plurality of objects. In some embodiments, the program further comprises: code that predicts a trajectory for each one of the plurality of objects; and code that determines whether the corresponding three-dimensional model for each one of the plurality of objects overlaps with another corresponding three-dimensional model for another one of the plurality of objects at a point along the trajectory. In some embodiments, the corresponding three-dimensional model comprises a mesh of polygons. The program further comprises code that halts a movement of the radiation therapy machine in response to a determination that the corresponding three-dimensional model for at least one of the plurality of objects in the depth map overlaps with another corresponding three-dimensional model for another one of the plurality of objects.

Disclosed are various embodiments for a system, comprising: at least one three-dimensional camera; at least one computing device in data communication with the at least one three-dimensional camera; and an application executed in the at least one computing device, the application comprising: logic that obtains a depth map produced by the at least one three-dimensional camera; logic that identifies a plurality of objects in the depth map, wherein the plurality of objects comprise a radiation therapy machine and a patient; logic that generates a corresponding three-dimensional model for each one of the plurality of objects; and logic that determines, based at least in part on the depth map, whether the corresponding three-dimensional model for each one of the plurality objects overlaps with another corresponding three-dimensional model for another one of the plurality objects. In some embodiments, the application further comprises: logic that obtains an updated depth map produced by the at least one three-dimensional camera; logic that identifies the plurality of objects in the updated depth map; and logic that determines, based at least in part on the updated depth map, whether the corresponding three-dimensional model for each one of the plurality of objects overlaps with another corresponding three-dimensional model for another one of the plurality of objects. In some embodiments, application further comprises: logic that predicts a trajectory for each one of the plurality of objects; and logic that determines whether the corresponding three-dimensional model for each one of the plurality of objects overlaps with another corresponding three-dimensional model for another one of the plurality of objects at a point along the trajectory. In some embodiments, the corresponding three-dimensional model comprises a mesh of polygons. In some embodiments, the application further comprises logic that halts a movement of the radiation therapy machine in response to a determination that the corresponding three-dimensional model for at least one of the plurality of objects in the depth map overlaps with another corresponding three-dimensional model for another one of the plurality of objects.

Disclosed are various embodiments for a computer-implemented method, comprising: obtaining, via a computing device, a depth map produced by at least one three-dimensional camera; identifying, via the computing device, a plurality of objects in the depth map, wherein the plurality of objects comprise a radiation therapy machine and a patient; generating, via the computing device, a corresponding three-dimensional model for each one of the plurality of objects; and determining, via the computing device, whether the corresponding three-dimensional model for each one of the plurality objects overlaps with another corresponding three-dimensional model for another one of the plurality objects. In some embodiments, the computer-implemented method further comprises: obtaining, via the computing device, an updated depth map produced by the at least one three-dimensional camera; identifying, via the computing device, the plurality of objects in the updated depth map; and determining, via the computing device, whether the corresponding three-dimensional model for each one of the plurality of objects overlaps with another corresponding three-dimensional model for another one of the plurality of objects. In some embodiments, the computer-implemented method further comprises: predicting, via the computing device, a trajectory for each one of the plurality of objects; and determining, via the computing device, whether the corresponding three-dimensional model for each one of the plurality of objects overlaps with another corresponding three-dimensional model for another one of the plurality of objects at a point along the trajectory. In some embodiments, the corresponding three-dimensional model comprises a mesh of polygons. In some embodiments, the computer-implemented method further comprises halting, via the computing device, a movement of the radiation therapy machine in response to a determination that the corresponding three-dimensional model for at least one of the plurality of objects in the depth map overlaps with another corresponding three-dimensional model for another one of the plurality of objects.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is a drawing of an example of a treatment system according to various embodiments of the present disclosure.

FIG. 2 is a drawing of a networked environment according to various embodiments of the present disclosure.

FIG. 3 is a flowchart illustrating one example of functionality implemented as portions of an application executed in a computing device in the networked environment of FIG. 2 according to various embodiments of the present disclosure.

FIG. 4 is a schematic block diagram that provides one example illustration of a computing environment employed in the networked environment of FIG. 1 according to various embodiments of the present disclosure.

### DETAILED DESCRIPTION

Disclosed are various embodiments for predicting and avoiding collisions between patients and radiation therapy machines during treatment. At least one three-dimensional imaging device located in the treatment area images a patient being treated by a radiation therapy machine and/or similar apparatus. The three-dimensional imaging device then sends a point cloud, depth map, or similar data to a computing device. The computing device identifies objects, such as the radiation therapy machine, the patient, and/or other objects in the treatment area, based at least in part on the point cloud and/or depth map. The computing device then generates a three-dimensional model for each identified object. Subsequently, the computing device determines whether two or more of the three-dimensional models overlap, indicating a collision between two or more corresponding objects. The computing device can further track the position, speed, and/or direction of movement of individual objects as updated depth data is received from the three-dimensional imaging device. Based at least in part on the accumulated trajectory data, future positions, speeds, and/or directions of movement for individual objects can be predicted. Based on the predicted trajectory data, the computing device can predict whether two or more of the corresponding three-dimensional models will overlap, indicating a collision between the corresponding objects, such as a collision between a radiation treatment machine and a patient.

In the following discussion, a general description of the system and its components is provided, followed by a discussion of the operation of the same.

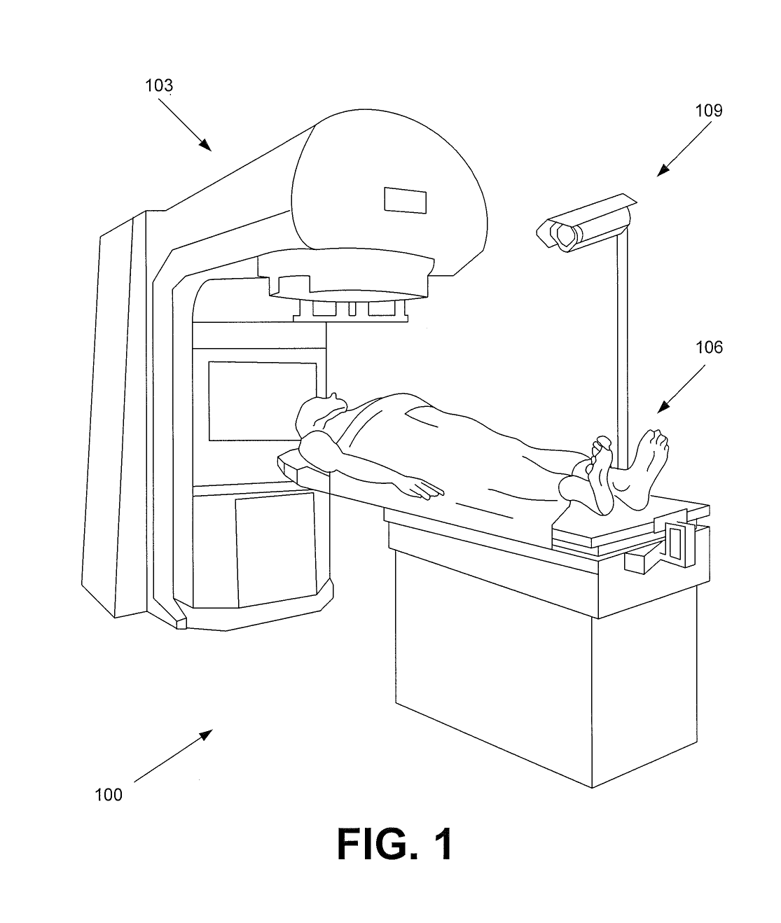

With reference to FIG. 1, shown is an example of a configuration of a treatment system 100 for various embodiments of the present disclosure. The treatment system can make use of a radiation treatment machine 103 or similar device positioned near a patient 106. The radiation treatment machine 103 and the patient 106 can be positioned within the field of view of a three-dimensional imaging device 109, such as a three-dimensional camera. In some embodiments, multiple three-dimensional imaging devices 109 can be positioned around the radiation treatment machine 103 and/or patient.

The radiation treatment machine 103 can comprise any one of a number of devices designed to irradiate portions or locations of a patient 106 for medical and/or therapeutic purposes. The radiation treatment machine 103 can be moved with respect to the patient 106 or can move components, such as an arm or gantry, around the patient 106 during the course of a treatment. In some embodiments, the radiation treatment machine 103 can be self-propelled, while in other embodiments, the radiation treatment machine 103 can be manually repositioned. Further, some embodiments can make use of self-propelled and manual positioning.

The three-dimensional imaging device 106 can include any camera and/or similar apparatus capable of generating a point cloud, depth map, and/or other three-dimensional representation of the field of view of the three-dimensional imaging device 106. For example, the three-dimensional imaging device 106 can include a laser or infrared projector combined with a sensor to generate three-dimensional images.

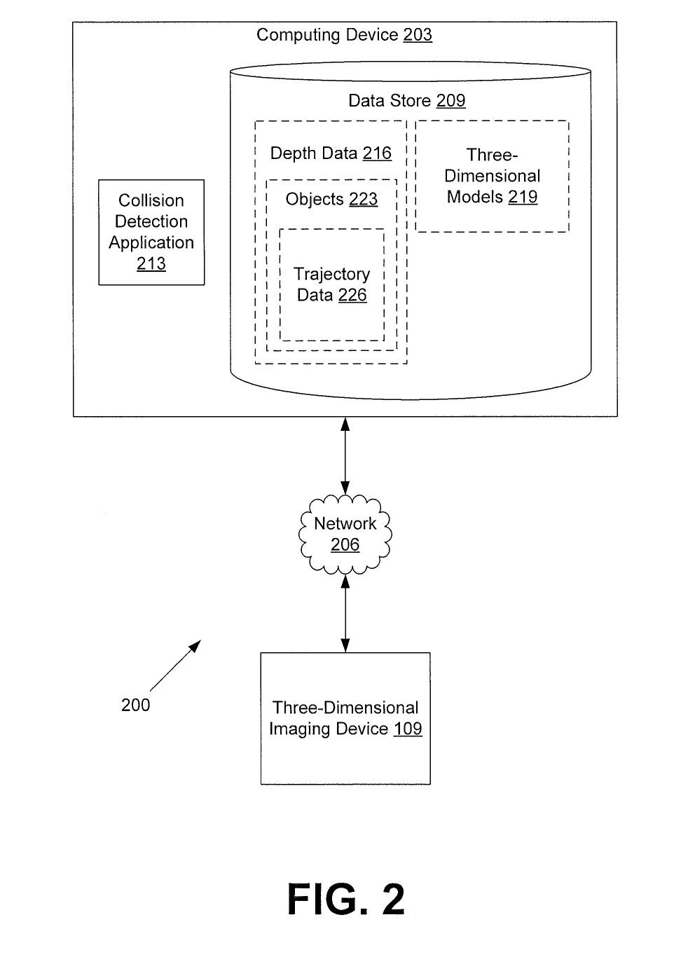

With reference to FIG. 2, shown is a networked environment 200 according to various embodiments. The networked environment 200 includes a computing device 203 and a three-dimensional imaging device 109 which are in data communication with each other via a network 206. The network 206 includes, for example, the Internet, intranets, extranets, wide area networks (WANs), local area networks (LANs), wired networks, wireless networks, or other suitable networks, etc., or any combination of two or more such networks. For example, such networks can comprise satellite networks, cable networks, Ethernet networks, and other types of networks.

The computing device 203 can comprise, for example, a server computer or any other system providing computing capability. Alternatively, the computing device 203 can be one of plurality of computing devices that can be arranged, for example, in one or more server banks or computer banks or other arrangements. Such computing devices can be located in a single installation or can be distributed among many different geographical locations. For example, the computing device 203 can be one of a plurality of computing devices that together can comprise a hosted computing resource, a grid computing resource and/or any other distributed computing arrangement. In some cases, the computing device 203 can correspond to an elastic computing resource where the allotted capacity of processing, network, storage, or other computing-related resources can vary over time.

Various applications and/or other functionality can be executed in the computing device 203 according to various embodiments. Also, various data is stored in a data store 209 that is accessible to the computing device 203. The data store 209 can be representative of a plurality of data stores 209 as can be appreciated. The data stored in the data store 209, for example, is associated with the operation of the various applications and/or functional entities described below.

The components executed on the computing device 203, for example, include the collision detection application 213, and other applications, services, processes, systems, engines, or functionality not discussed in detail herein. The collision detection application 213 is executed to analyze data received from the three-dimensional imaging device 109 to predict collisions between objects in the treatment system 100 (FIG. 1), such as a collision between the radiation treatment machine 103 (FIG. 1) and the patient 106 (FIG. 1). If a collision is predicted, the collision detection application 213 can also halt treatment, sound an alarm, send an error message, and/or take other appropriate action.

The data stored in the data store 209 includes, for example, depth data 216, three-dimensional models 219, and potentially other data. The depth data 216 includes three-dimensional data received from one or more three-dimensional imaging devices 109. The depth data 216 can, for example, correspond to a depth map, point cloud, and/or similar data. The depth data can also include data related to one or more objects 223. The objects 223 can correspond to objects identified in the depth data 216 by the collision detection application 213 and/or the three-dimensional imaging device 109. The three-dimensional models 219 correspond to wire frame models, mesh models, and/or other three-dimensional representations of the objects 223 identified in the depth data 216. Further, each object 223 can have trajectory data 226 associated with it. The trajectory data 226 can include data related to the current position, speed, and/or direction of movement of the object 223, as well as a history of previous positions, speeds, and/or directions of movement at previous points in time. In some embodiments, the trajectory data 226 can also include predicted or anticipated positions, speeds, and/or directions of movement at future points in time.

Next, a general description of the operation of the various components of the networked environment 100 is provided. To begin, the three-dimensional imaging device 109 images a patient 106 being treated by a radiation therapy machine 103. The three-dimensional imaging device 109 then sends depth data 216 to the computing device 203. The collision detection application 213 identifies objects 223, such as the radiation therapy machine 103, the patient 106, and/or other objects, within the depth data 216. The collision detection application 213 then generates a three-dimensional model 219 for each identified object 223. Subsequently, the collision detection application 213 determines whether two or more of the three-dimensional models 219 overlap, indicating a collision between two or more objects 223. The collision detection application 213 can further track the position, speed, and/or direction of movement of individual objects 223 as the collision detection application 213 processes updated depth data 216 received from the three-dimensional imaging device 109. Based at least in part on the accumulated trajectory data 226, the collision detection application 213 can predict future positions, speeds, and/or directions of movement for individual objects 223. Based on the predicted trajectory data 226, the collision detection application 213 can predict whether two or more of the corresponding three-dimensional models 219 will overlap, indicating a collision between the corresponding objects 223, such as a collision between a radiation treatment machine 103 and a patient 106.

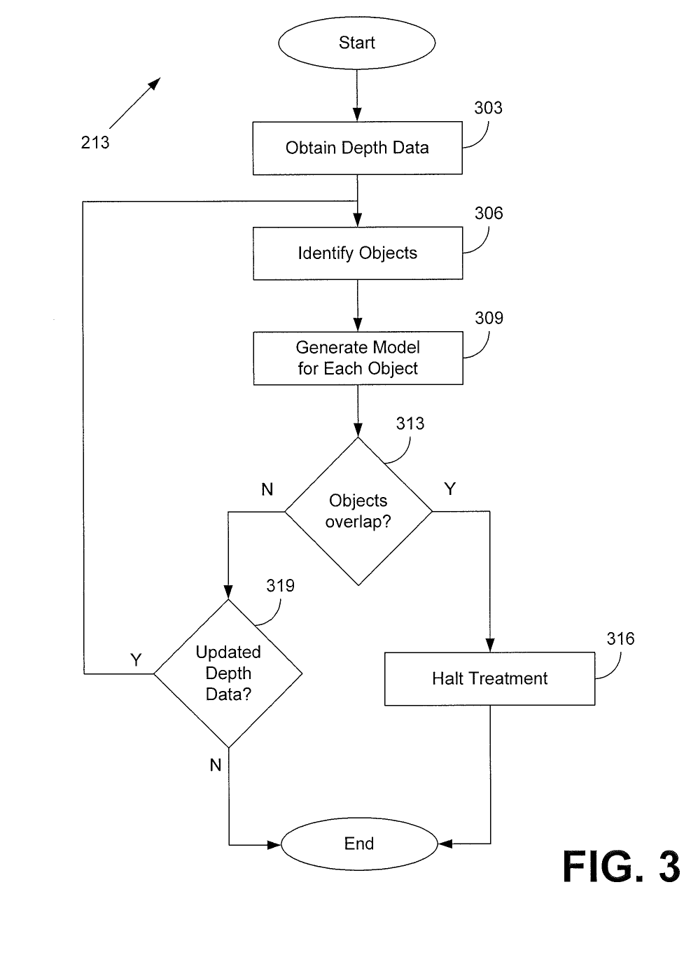

Referring next to FIG. 3, shown is a flowchart that provides one example of the operation of a portion of the collision detection application 213 according to various embodiments. It is understood that the flowchart of FIG. 3 provides merely an example of the many different types of functional arrangements that can be employed to implement the operation of the portion of the collision detection application 213 as described herein. As an alternative, the flowchart of FIG. 3 can be viewed as depicting an example of elements of a method implemented in the computing device 203 (FIG. 2) according to one or more embodiments.

Beginning with box 303, the collision detection application 213 obtains depth data 216 (FIG. 2) from a three-dimensional imaging device 109 (FIGS. 1 and 2) and stores the depth data 216 in a data store 209 (FIG. 2). The depth data 216 may be obtained through a network connection 206 (FIG. 2).

Proceeding next to box 306, the collision detection application 213 identifies one or more objects 223 within the depth data 216, such as a radiation therapy machine 103 (FIG. 1) or a patient 106 (FIG. 1). Objects 223 may be identified using one or more of a number of computer vision approaches, such as edge detection, greyscale matching, gradient matching, and/or various other approaches.

Moving on to box 309, the collision detection application 213 generates a three-dimensional model 219 for each object 223 identified. The collision detection application 213 may, for example, generate a wire-frame model, a mesh model, and/or various other types of three-dimensional models 219.

Referring next to box 313, the collision detection application 213 determines whether any two three-dimensional models 219 overlap. The collision detection application 213 may use, or example, a hierarchical bounded box approach, where each of the three-dimensional models 219 is divided into a hierarchy of boxes. Collisions may then be determined by identifying where two or more of the bounded boxes overlap and/or intersect. Other approaches may also be used. If it is determined that two or more objects 223 have collided or are about to collide, execution proceeds to box 316. If none of the objects 223 overlap, then execution proceeds to box 319.

Proceeding next to box 316, the collision detection application 213 halts treatment of the patient 106. Treatment may be halted using one or more of several approaches. For example, the collision detection application 213 may send a message or instruction to the radiation treatment machine 103 to stop treatment and cease moving, or to stop treatment and return to a neutral or “safe” position. The collision detection application 213 may, in some embodiments, initiate an alarm or send a message to a treating physician or technician, such as by causing a message to be rendered on the control panel of the radiation treatment machine 103. After treatment is halted, execution ends.

Moving on to box 319, the collision detection application 213 determines whether new depth data 216, such as an updated depth map or similar image, has been provided by the three-dimensional imaging device 109. If new depth data 216 has been received, execution loops back to box 306. In some embodiments, the collision detection application 213 may also update trajectory data 226 of individual objects 223 at this stage. If no additional depth data 216 has been received, then execution subsequently ends. However, it is understood that, in some embodiments, the collision detection application 213 may wait a brief amount of time to receive updated depth data 216 before ending execution.

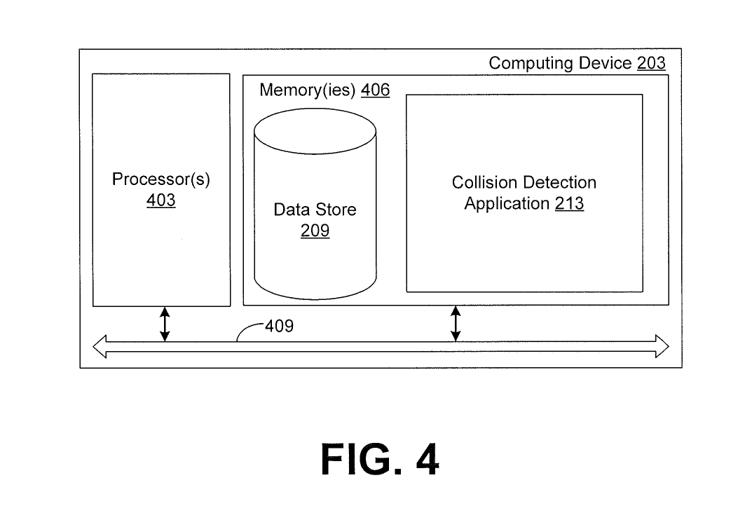

With reference to FIG. 4, shown is a schematic block diagram of the computing device 203 according to an embodiment of the present disclosure. The computing device 203 can be one of one or more computing devices. The computing device 203 includes at least one processor circuit, for example, having a processor 403 and a memory 406, both of which are coupled to a local interface 409. To this end, a computing device 203 can comprise, for example, at least one server computer or like device. The local interface 409 can comprise, for example, a data bus with an accompanying address/control bus or other bus structure as can be appreciated.

Stored in the memory 406 are both data and several components that are executable by the processor 403. In particular, stored in the memory 406 and executable by the processor 403 are the collision detection application 213, and potentially other applications. Also stored in the memory 406 can be a data store 209 and other data. In addition, an operating system can be stored in the memory 406 and executable by the processor 403.

It is understood that there can be other applications that are stored in the memory 406 and are executable by the processor 403 as can be appreciated. Where any component discussed herein is implemented in the form of software, any one of a number of programming languages can be employed such as, for example, C, C++, C#, Objective C, Java®, JavaScript®, Perl, PHP, Visual Basic®, Python®, Ruby, Flash®, or other programming languages.

A number of software components are stored in the memory 406 and are executable by the processor 403. In this respect, the term “executable” means a program file that is in a form that can ultimately be run by the processor 403. Examples of executable programs can be, for example, a compiled program that can be translated into machine code in a format that can be loaded into a random access portion of the memory 406 and run by the processor 403, source code that can be expressed in proper format such as object code that is capable of being loaded into a random access portion of the memory 406 and executed by the processor 403, or source code that can be interpreted by another executable program to generate instructions in a random access portion of the memory 406 to be executed by the processor 403, etc. An executable program can be stored in any portion or component of the memory 406 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 406 is defined herein as including both volatile and nonvolatile memory and data storage components. Volatile components are those that do not retain data values upon loss of power. Nonvolatile components are those that retain data upon a loss of power. Thus, the memory 406 can comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM can comprise, for example, static random access memory (SRAM), dynamic random access memory (DRAM), or magnetic random access memory (MRAM) and other such devices. The ROM can comprise, for example, a programmable read-only memory (PROM), an erasable programmable read-only memory (EPROM), an electrically erasable programmable read-only memory (EEPROM), or other like memory device.

Also, the processor 403 can represent multiple processors 403 and/or multiple processor cores and the memory 406 can represent multiple memories 406 that operate in parallel processing circuits, respectively. In such a case, the local interface 409 can be an appropriate network that facilitates communication between any two of the multiple processors 403, between any processor 403 and any of the memories 406, or between any two of the memories 406, etc. The local interface 409 can comprise additional systems designed to coordinate this communication, including, for example, performing load balancing. The processor 403 can be of electrical or of some other available construction.

Although the collision detection application 213, and other various systems described herein can be embodied in software or code executed by general purpose hardware as discussed above, as an alternative the same can also be embodied in dedicated hardware or a combination of software/general purpose hardware and dedicated hardware. If embodied in dedicated hardware, each can be implemented as a circuit or state machine that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowchart of FIG. 3 shows the functionality and operation of an implementation of portions of the collision detection application 213. If embodied in software, each block can represent a module, segment, or portion of code that comprises program instructions to implement the specified logical function(s). The program instructions can be embodied in the form of source code that comprises human-readable statements written in a programming language or machine code that comprises numerical instructions recognizable by a suitable execution system such as a processor 403 in a computer system or other system. The machine code can be converted from the source code, etc. If embodied in hardware, each block can represent a circuit or a number of interconnected circuits to implement the specified logical function(s).

Although the flowchart of FIG. 3 shows a specific order of execution, it is understood that the order of execution can differ from that which is depicted. For example, the order of execution of two or more blocks can be scrambled relative to the order shown. Also, two or more blocks shown in succession in FIG. 3 can be executed concurrently or with partial concurrence. Further, in some embodiments, one or more of the blocks shown in FIG. 3 can be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein, including the collision detection application 213, that comprises software or code can be embodied in any non-transitory computer-readable medium for use by or in connection with an instruction execution system such as, for example, a processor 403 in a computer system or other system. In this sense, the logic can comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium can be a random access memory (RAM) including, for example, static random access memory (SRAM) and dynamic random access memory (DRAM), or magnetic random access memory (MRAM). In addition, the computer-readable medium can be a read-only memory (ROM), a programmable read-only memory (PROM), an erasable programmable read-only memory (EPROM), an electrically erasable programmable read-only memory (EEPROM), or other type of memory device.

Further, any logic or application described herein, including the collision detection application 213, can be implemented and structured in a variety of ways. For example, one or more applications described can be implemented as modules or components of a single application. Further, one or more applications described herein can be executed in shared or separate computing devices or a combination thereof. For example, a plurality of the applications described herein can execute in the same computing device 203, or in multiple computing devices in the same computing device 203. Additionally, it is understood that terms such as “application,” “service,” “system,” “engine,” “module,” and so on can be interchangeable and are not intended to be limiting.

Disjunctive language such as the phrase “at least one of X, Y, or Z,” unless specifically stated otherwise, is otherwise understood with the context as used in general to present that an item, term, etc., can be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications can be made to the above-described embodiment(s) without departing substantially from the spirit and principles of the disclosure. All such modifications and variations are intended to be included herein within the scope of this disclosure and protected by the following claims.

Therefore, the following is claimed:

1. A non-transitory computer-readable medium embodying a program executable in at least one computing device, comprising:
code that obtains a depth map produced by a three-dimensional camera;
code that identifies a plurality of objects in the depth map, wherein the plurality of objects comprise a radiation therapy machine and a patient;
code that generates a corresponding three-dimensional model for each one of the plurality of objects; and
code that determines whether the corresponding three-dimensional model for each one of the plurality objects overlaps with another corresponding three-dimensional model for another one of the plurality objects.

2. The non-transitory computer-readable medium of claim 1, wherein the program further comprises:
code that obtains an updated depth map produced by the at least one three-dimensional camera;
code that identifies the plurality of objects in the updated depth map; and
code that determines, based at least in part on the updated depth map, whether the corresponding three-dimensional model for each one of the plurality of objects overlaps with another corresponding three-dimensional model for another one of the plurality of objects.

3. The non-transitory computer-readable medium of claim 1, wherein the program further comprises:
code that predicts a trajectory for each one of the plurality of objects; and
code that determines whether the corresponding three-dimensional model for each one of the plurality of objects overlaps with another corresponding three-dimensional model for another one of the plurality of objects at a point along the trajectory.

4. The non-transitory computer-readable medium of claim 1, wherein the corresponding three-dimensional model comprises a mesh of polygons.

5. The non-transitory computer-readable medium of claim 1, wherein the program further comprises code that halts a movement of the radiation therapy machine in response to a determination that the corresponding three-dimensional model for at least one of the plurality of objects in the depth map overlaps with another corresponding three-dimensional model for another one of the plurality of objects.

6. A system, comprising:
at least one three-dimensional camera;
at least one computing device in data communication with the at least one three-dimensional camera; and
an application executed in the at least one computing device, the application comprising:
logic that obtains a depth map produced by the at least one three-dimensional camera;
logic that identifies a plurality of objects in the depth map, wherein the plurality of objects comprise a radiation therapy machine and a patient;
logic that generates a corresponding three-dimensional model for each one of the plurality of objects; and
logic that determines, based at least in part on the depth map, whether the corresponding three-dimensional model for each one of the plurality objects overlaps with another corresponding three-dimensional model for another one of the plurality objects.

7. The system of claim 6, wherein the application further comprises:
logic that obtains an updated depth map produced by the at least one three-dimensional camera;
logic that identifies the plurality of objects in the updated depth map; and
logic that determines, based at least in part on the updated depth map, whether the corresponding three-dimensional model for each one of the plurality of objects overlaps with another corresponding three-dimensional model for another one of the plurality of objects.

8. The system of claim 6, wherein the application further comprises:
logic that predicts a trajectory for each one of the plurality of objects; and
logic that determines whether the corresponding three-dimensional model for each one of the plurality of objects overlaps with another corresponding three-dimensional model for another one of the plurality of objects at a point along the trajectory.

9. The system of claim 6, wherein the corresponding three-dimensional model comprises a mesh of polygons.

10. The system of claim 6, wherein the application further comprises logic that halts a movement of the radiation therapy machine in response to a determination that the corresponding three-dimensional model for at least one of the plurality of objects in the depth map overlaps with another corresponding three-dimensional model for another one of the plurality of objects.

11. A computer-implemented method, comprising:
obtaining, via a computing device, a depth map produced by at least one three-dimensional camera;
identifying, via the computing device, a plurality of objects in the depth map, wherein the plurality of objects comprise a radiation therapy machine and a patient;
generating, via the computing device, a corresponding three-dimensional model for each one of the plurality of objects; and
determining, via the computing device, whether the corresponding three-dimensional model for each one of the plurality objects overlaps with another corresponding three-dimensional model for another one of the plurality objects.

12. The computer-implemented method of claim 11, further comprising:
obtaining, via the computing device, an updated depth map produced by the at least one three-dimensional camera;
identifying, via the computing device, the plurality of objects in the updated depth map; and
determining, via the computing device, whether the corresponding three-dimensional model for each one of the plurality of objects overlaps with another corresponding three-dimensional model for another one of the plurality of objects.

13. The computer-implemented method of claim 11, further comprising:
predicting, via the computing device, a trajectory for each one of the plurality of objects; and
determining, via the computing device, whether the corresponding three-dimensional model for each one of the plurality of objects overlaps with another corresponding three-dimensional model for another one of the plurality of objects at a point along the trajectory.

14. The computer-implemented method of claim 11, wherein the corresponding three-dimensional model comprises a mesh of polygons.

15. The computer-implemented method of claim 11, further comprising halting, via the computing device, a movement of the radiation therapy machine in response to a determination that the corresponding three-dimensional model for at least one of the plurality of objects in the depth map overlaps with another corresponding three-dimensional model for another one of the plurality of objects.
